SEQUENCE LISTING (13 sequences)

SEQ ID NO: 1 - DNA, 354 bp, Homo sapiens
tgtgtaaaggccccctatcctatccgcaaacagatgtgttcttaatttgcttttcccttgtgagtcctgcatcatttgaaaatgtccgtgcaaagtggtatcctgaggtgcggcaccactgtcccaacactcccatcatcctagtgggaactaaacttgatcttagggatgataaagacacgatcgagaaactgaaggagaagaagctgactcccatcacctatccgcagggtctagccatggctaaggagattggtgctgtaaaatacctggagtgctcggcgctcacacagcgaggcctcaagacagtgtttgacgaagcgatccgagcagtcctctgcccgcctcacaatc

SEQ ID NO: 2 - DNA, 344 bp, Homo sapiens
tgatggaggcgggcagaggactgctcggatcgcttcgtcaaacactgtcttgaggcctcgctgggggaacggccaacactccagggattttacaggaccaatctccttaaccatggctaaaacctggggataggggatgggaggcagcttcttctccttcaggttctcgatcggggctttatcatccctaagaacaaggttaattcccactaagatgatgggagtggtgggacaggggtggcccacctcaagataccactttggacggacattttccaatggtggaggactcccaagggaaaagcaaattaagaacacatctggttgcggataagataaggggc

SEQ ID NO: 3 - DNA, 354 bp, Homo sapiens; identical to SEQ ID NO: 1

SEQ ID NO: 4 - DNA, 354 bp, Homo sapiens
tgtggttaggcgggcagaggactgctcggatcgcttcgtcaaacactgtcttgaggcctcgctgtgtgagcgccgagcactccaggtattttacagcaccaatctccttagccatggctagaccctgcggataggtgatgggagtcagcttcttctccttcagtttctcgatcgtgtctttatcatccctaagatcaagtttagttcccactaggatgatgggagtgttgggacagtggtgccgcacctcaggataccactttgcacggacattttcaaatgatgcaggactcacaagggaaaagcaaattaagaacacatctgtttgcggataggatagggggcctaccgatg

SEQ ID NO: 5 - DNA, 348 bp, Homo sapiens
tgtggggaggcgggcagaggactgctcggatcgcttcgtcaaacactgtcttgaggcctcgctgtgtgagcgccgagcactccaggtattttacagcaccaatctccttagccatggctagaccctgcggataggtgatgggagtcagcttcttctccttcagtttctcgatcgtgtctttatcatccctaagatcaagtttagttcccactaggatgatgggagtgttgggacagtggtgccgcacctcaggataccactttgcacggacattttcaaatgatgcaggactcacaagggaaaagcaaattaagaacacatctgtttgcggataggatagggggcctc

SEQ ID NO: 6 - DNA, 356 bp, Homo sapiens
gtatgggaggccccctatcctatccgcaaacagatgtgtacttaatttgcttttcccttgtgagtcctgaatcatttgaaaatgtccgtgcaaagtgtcatcctgaggtgcggcaccactgtcccaacactcccatcatcctagtgggaactaaacttgatcttaaggatgataaagacacgatcgagaaactgaagggagaagaagctgactcccatcacctatccggagggcctaggcatggctaaggagattggtgctgccaaaatacctggagtgctcggcgctcacacagcaagggctcaagacagtgtttgacgaagcgatccgagcagtcctctgcccgcctagcacag

SEQ ID NO: 7 - DNA, 353 bp, Homo sapiens
attgttgaggcgggcagaggactgctcggatcgcttcgtcaacactgtcttgaggcctcgctgtgtgagcgccgagcactccaggtattttacagcaccaatctccttagccatggctagaccctgcggataggtgatgggagtcagcttcttctccttcagtttctcgatcgtgtctttatcatccctaagatcaggtttagttcccactaggatgatgggagtgttgggacagtggtgccgcacctcaggataccactttgcacggacattttcaaatgatgcaggactcacaagggaaaagcaaattaagaacacatctgtttgcggataggatagggggcctttccacc

SEQ ID NO: 8 - DNA, 544 bp, Homo sapiens
aggccatcaagtgtgtggtggtgggagacggagctgtatgtaaaacttgcctactgatcagttacacaaccaatgcatttcctggagaatatatccctactgtctttgacaattattctgccaatgttatggtagatggaaaaccggtgaatctgggcttatgggatacagctggacaagaatattatgacagattacgccccctatcctatccgcaaacagatgtgttcttaatttgcttttcccttgtgagtcctgcatcatttgaaaatgtccgtgcaaagtggtatcctgaggtgcggcaccactgtcccaacactcccatcatcctagtgggaactaaacttgatcttagggatgataaagacacgatcgagaaactgaaggagaagaagctgactcccatcacctatccgcagggtctagccatggctaaggagattggtgctgtaaaatacctggagtgctcggcgctcacacagcgaggcctcaagacagtgtttgacgaagcgatccgagcagtcctctgcccgcctcacaatca

SEQ ID NO: 9 - DNA, 22 bp, Artificial
  Oligonucleotide F1 (242-263)
atgcaggccatcaagtgtgtgg

SEQ ID NO: 10 - DNA, 19 bp, Artificial
  Oligonucleotide R1 (1012-1030)
tggcattgagtgcgaaggc

SEQ ID NO: 11 - DNA, 19 bp, Artificial
  Oligonucleotide F2 (460-483)
aaagacaagccgattgccg

SEQ ID NO: 12 - DNA, 240 bp, Homo sapiens
cagtgacttagcttctacacctgtgactaaccattttcattccattctacagctttgacaattattctgccaatgttatggtagatggaaaaccggtgaatctgggcttatgggatacagctggacaagaagattatgacagattacgccccctatcctatccgcaaacagtaaggattgcagctgacttttaatgtgtcttttagagtatataattctcgagcgcttaattagtgcatg

SEQ ID NO: 13 - protein, 19 aa, Homo sapiens
VGETYGKDITSRGKDKPIA

(19) United States
(12) Patent Application Publication
(10) Pub. No.: US 2009/0285820 A1
(43) Pub. Date: Nov. 19, 2009

(54) Methods of Treating Degenerative Disorders With Rac 1B Inhibitor

(76) Inventors: Laurent Desire, Paris (FR); Virginie Picard, Paris (FR); Séverine Coutadeur, Vitry sur Seine (FR); Fabien Schweighoffer, Nogent Sur Marne (FR)

Correspondence Address: NIXON & VANDERHYE, PC, 901 NORTH GLEBE ROAD, 11TH FLOOR, ARLINGTON, VA, 22203, US

(21) Appl. No.: 12/223,520

(22) PCT Filed: Jan. 30, 2007
(86) PCT No.: PCT/EP2007/050872

§ 371 Date: Nov. 18, 2008

Related U.S. Application Data
(60) Provisional application No. 60/743,199, filed on Feb. 1, 2006.

(51) Int. Cl. A61K 39/395; C12Q 1/68; G01N 33/53; C12Q 1/34; C07K 7/08; C07K 16/18; C07H 21/00

(52) U.S. Cl. 424/139.1; 435/6; 435/7.8; 435/18; 530/326; 530/387.9; 536/23.1

(57) ABSTRACT

The invention relates to compositions and methods for treating degenerative disorders. More particularly, the invention relates to methods of treating amyloïd beta peptide-related disorders, particularly Alzheimer's disease, using Rac1b inhibitors. The invention may be used in mammalian subjects, particularly human subjects, at various stages of the disease, including disease onset. The invention also provides methods of producing, identifying, selecting or optimising compounds for use in the treatment of amyloïd beta peptide-related disorders, based on a determination of the ability of a test compound to inhibit Rac1b.

The invention relates to compositions and methods for treating degenerative disorders using Rac1b inhibitors. More particularly, the invention relates to methods of treating amyloïd beta peptide-related disorders, particularly Alzheimer's disease. The invention may be used in mammalian subjects, particularly human subjects, at various stages of the disease, including disease onset. The invention also provides methods of producing, identifying, selecting or optimising compounds for use in the treatment of degenerative disorders, based on a determination of the ability of a test compound to inhibit Rac1b. The invention also encompasses methods of detecting the presence, stage or nature of a degenerative disorder in a subject, comprising assessing the presence or (relative) amount of Rac1b in a sample from said subject.

Alzheimer's disease (AD) is a progressive neurodegenerative disease primarily characterized by behaviour and cognitive defects. The brains of AD patients are characterized by the accumulation of extracellular plaques beta-amyloid (Abeta) peptides and of intracellular neurofibrillary tangles containing hyperphosphorylated forms of tau.

It has been proposed that, in the initial phase of disease development, Abeta deposition and hyperphosphorylation of tau may function as compensatory responses and downstream adaptations against oxidative stress to ensure neuronal cells survival. However, during the progression of the disease, the antioxidant activity of both agents evolves into pro-oxidant activity representing a typical gain-of-function transformation which can involve conformation changes and a decrease in clearance mechanisms.

The present invention describes new findings that define a new target for therapeutic intervention to control oxidative stress and therefore to improve neuron viability as well as to control Abeta production and tau hyperphosphorylation.

We have applied DATAS to brain biopsies of patients with Alzheimer's disease and controls. DATAS is a patented gene profiling technology (U.S. Pat. No. 6,251,590), which allows the analysis of transcripts that are differentially spliced between two physiopathological situations. This analysis revealed a deregulation of the splicing of exon3b of the Rac1 gene (example 1), leading to an overexpression of a particular splicing isoform, Rac1b, in diseased brains. The Rac1 gene can lead to two alternatively spliced mRNA encoding two proteins, Rac1a and Rac1b (FIG. 1). Rac1b is a constitutively activated version of Rac1a and is mainly involved in the activation of the oxidative stress cascade. The unexpected finding that Rac1b, constitutively activated, is overexpressed in diseased tissues is therefore compatible with the increase of oxidative stress in the brain of AD patients as compared to healthy individuals.

This represents the first evidence for a deregulation and increased expression of Rac1b in the brain of patients having degenerative diseases.

To confirm the existence and relevance of this deregulation, we have used various concentrations of Abeta25-35 on rat cortical cells to test Abeta-induced changes in the alternative splicing of Rac1 gene. The beta amyloid peptide Abeta 42 is the main constituent of senile plaques found in AD brain. Abeta peptide acts on neuronal cells to induce oxidative stress and an increase in intracellular free calcium content, which are necessary events in mediating Abeta toxicity as well as secondary excitotoxicity. Other mechanisms are increased phosphorylation of tau and induction of gene transcription. Abeta25-35 is a fragment of Abeta42 containing the aminoacids 25 to 35 that reproduces the toxic mechanisms of Aβ42. Rat cortical primary cultures are sensitive to Abeta intoxification which results in dose-dependent loss in cell viability. Our results, shown in Example 2, unexpectedly show that Abeta treatment induces, in a dose-dependent manner, the expression of Rac1b mRNA.

To further confirm the link between Abeta treatment, oxidative stress and regulation of the alternative splicing of Rac1 gene toward the Rac1b isoform, we also applied oxidative stress on SH-SY5Y neuroblastoma cells, by either t-butyl hydroperoxide (TBH) or 6-hydroxydopamine (6-OHDA) (example 3). Our results show that oxidative stress also induces, in a dose-dependent manner, the expression of Rac1b mRNA in this neuronal cell line.

Since Rac1b itself induces oxidative stress pathways, the induction of its expression after beta amyloid peptide treatment or after TBH or 6-OHDA treatment, is likely to amplify the stress on cells. This represents the first evidence that beta amyloid peptide, likely through oxidative stress, can induce Rac1b expression in neuronal cells.

This invention thus presents the first evidence that Rac1b can be considered as a therapeutic and diagnostic target for neurodegenerative disease. In particular, the invention shows that Rac1b inhibitors represent a new class of molecules for use in the treatment of degenerative disorders. The invention further shows that Rac1b represents a valuable target for the screening or optimisation of (new) chemical entities for treating degenerative disorders.

Accordingly, one aspect of the invention relates to a method of treating a degenerative disorder in a mammalian subject, comprising administering to a subject in need thereof an effective amount of a Rac1b inhibitor.

The invention also relates to the use of a Rac1b inhibitor for the manufacture of a pharmaceutical composition for treating a degenerative disease. A further aspect of this invention is a method of inhibiting the generation of an amyloïd beta peptide in a mammalian subject, comprising administering to a subject in need thereof an effective amount of a Rac1b inhibitor.

A further aspect of this invention is a method of inhibiting the generation of an amyloïd beta peptide in a mammalian subject without substantially altering the Notch cleavage or BACE activity, comprising administering to a subject in need thereof an effective amount of a Rac1b inhibitor.

A further object of this invention relates to a method of treating a degenerative disorder in a mammalian subject, comprising administering to a subject in need thereof an amount of an inhibitory nucleic acid compound effective at reducing Rac1b expression (e.g., transcription, splicing and/or translation) in said subject.

A further object of this invention relates to a method of treating a degenerative disorder in a mammalian subject, comprising administering to a subject in need thereof an amount of an antibody effective at reducing Rac1b activity in said subject.

A further object of this invention relates to a method of treating a degenerative disorder in a mammalian subject, comprising administering to a subject in need thereof an effective amount of small drug compound effective at reducing Rac1b activity in said subject.

For use in the present invention, the Rac1b inhibitors may be formulated in the presence of any pharmaceutically acceptable support or excipient, and they may be used either alone or in combination(s), optionally together with any other active agent(s) or treatment(s).

The invention may be used to treat various degenerative disorders, particularly amyloïd beta peptide-related disorders, including Alzheimer's disease, at various stage of the disorder, in any mammalian subject, preferably human subjects.

The present invention further relates to methods of detecting the presence or predisposition to oxidative stress comprising detecting, in a sample from a subject, the presence or (relative) amount of Rac1b, the presence of Rac1b being indicative of the presence or predisposition to oxidative stress.

The present invention further relates to methods of detecting the presence, stage or predisposition to a degenerative disorder in a subject, comprising detecting, in a sample from the subject, the presence or (relative) amount of Rac1b, the presence of Rac1b being indicative of the presence, stage or predisposition to said disorder.

A further object of this invention is a method of producing, identifying, selecting or optimising candidate compounds, comprising a step of determining whether a candidate compound can inhibit Rac1b.

The invention also relates to a method of producing, identifying, selecting or optimising candidate compounds for use in the treatment of degenerative disorders, the method comprising determining whether a test compound inhibits Rac1b, Rac1b inhibition being an indication that the test compound is a candidate compound for use in the treatment of degenerative disorders. Rac1b inhibition may be assessed in vitro, ex vivo or in vivo, using biological/immuno techniques which are known per se in the art. Preferably, the compounds are further assessed for their activity towards Notch cleavage, compounds which substantially do not alter Notch cleavage being preferred.

The invention also relates to antibodies that specifically bind Rac1b polypeptide, as well as to nucleic acid molecules that specifically bind Rac1b gene or RNA.

### LEGEND TO THE FIGURES

FIG. 1: Diagram of the organization of rac1 (NM_{—}006908) and rac1b (NM_{—}006908) genes. Arrows indicate PCR primers and 3b indicates the additional exon3b in rac1b.

FIG. 2: Induction of Rac1b by beta amyloid peptide. Rat primary cortical cells were treated with Abeta at the indicated dose for 24 h. Shown agarose gels represent the PCR amplification of rac1 and rac1b in the same cells.

FIG. 3: Induction of Rac1b by various oxidative stress: A: hydroperoxide t-butyl hydroperoxide (TBH); B: 6-hydroxydopamine (6-OHDA). Histograms represent cell viability of SH-SY5Y cells treated with TBH or 6-OHDA for 24 h at the indicated dose, as determined using a LDH assay. Shown agarose gels represent the PCR amplification of rac1 and rac1b in the same cells.

### DETAILED DESCRIPTION OF THE INVENTION

The invention stems, inter alia, from the unexpected discovery that the rac1/rac1b ratio is biased towards the appearance of a highly activated variant of Rac1, Rac1b, in brain tissue from subjects having degenerative disorders. The invention thus relates to compositions and methods using Rac1 as a target for therapeutic or diagnostic intervention for degenerative disorders, as well as for developing active compounds.

### Degenerative Disorder

The term degenerative disorder includes any neurodegenerative disorder, particularly Amyloïd beta peptide-related disorders. These include, specifically, all disorders which are caused or associated with an increased or abnormal production of an Amyloïd beta peptide, particularly of Aβ40 and/or Aβ42.

Alzheimer's disease (AD) is the most common neurodegenerative disorder marked by progressive loss of memory and cognitive ability. The pathology of AD is characterized by the presence of amyloid plaques, intracellular neurofibrillary tangles and pronounced cell death. The β-amyloid peptide (Aβ) is the main constituent of senile plaques found in AD brains. Overproduction, intracellular accumulation, aggregation, and deposition in brain of the 42-amino acid form of Aβ (Aβ42) is associated with early onset, familial AD. Furthermore, extracellular Aβ42 appears toxic to neurons in vitro and in vivo (reviewed in Selkoe, D. J. (2001) Physiol. Rev. 81, 741-766). Aβ is generated by proteolysis of an integral membrane protein, the amyloid precursor protein (APP) via at least two post-translational pathways. The amyloidogenic cleavage of APP is a sequential processing of APP initiated by β-secretase (BACE), which cleaves APP within the luminal domain or at the cell surface, generating the N terminus of Aβ. This cleavage generates several membrane bound proteolytic C-terminal fragments (CTFs), such as the 99 residue β-CTF (also called C99), as well as the secreted APP ectodomain sAPPβ. The C-terminus of Aβ is subsequently generated by intramembraneous cleavage of CTFs by γ-secretase, producing either Aβ40 or Aβ42. The cleavages at residues 40-42 are referred to as γ-cleavage and the cleavage at residues 49-52 are referred to as ε-cleavage. The nonamyloidogenic cleavage of APP, which precludes Aβ generation, is mediated by α-secretase, a disintegrin and metalloproteinase 10 (ADAM-10) and ADAM-17, in a reaction believed to occur primarily on the plasma membrane. This proteolytical cleavage by α-secretase occurs within the Aβ region and produces soluble APP (sAPPα), the dominant processing product and the residual membrane bound 10-kDa CTF (CTFα also called C83). Like C99, C83 is a substrate for γ-secretase which cleaves to generate the non amyloidogenic p3 fragment. APP is also a substrate of caspase activities that cleave its cytosolic domain.

Examples of such Amyloïd beta peptide-related disorders therefore include any disease or condition selected from the group consisting of Alzheimer's disease (e.g., for helping prevent or delay the onset of Alzheimer's disease, for helping to slow the progression of Alzheimer's disease, for treating patients with mild cognitive impairment (MCI) and preventing or delaying the onset of Alzheimer's disease in those who would progress from MCI to AD), Mild cognitive Impairment (MCI), Down's syndrome, Hereditary Cerebral Hemorrhage with Amyloidosis of the Dutch-Type, cerebral amyloid angiopathy and its potential consequences (e.g., single and recurrent lobar hemorrhages), degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease.

### Treatment

Within the context of this application, the terms “treatment” or “treating” include both therapeutic and prophylactic treatment. In particular, the compounds may be used at very early stages of a disease, or before early onset, or after significant progression thereof. The term “treatment” or “treating” designates in particular a reduction of the burden in a patient, such as preventing or delaying the onset of the disease or disease progression, restoring or increasing cognitive functions or memory in a subject, reducing oxidative stress, delaying APP processing, etc.

### Rac1b

Rac1 is a small GTP-binding protein from the Rho family, such as Rho and Cdc42. These small G proteins are activated by GTP/GDP exchange and regulate a wide variety of cellular functions such as gene expression, cytoskeletal reorganization, and vesicle/secretory trafficking. The activated CDC42 or Rac then activates the PAK Ser/Thr kinase family. Recent studies showed the participation of Rho in the formation of stress fibers, while activated Cdc42 induces the formation of filopodia, thin fingerlike extensions containing actin bundles and Rac regulates the formation of lamellipodia or ruffles, curtain-like extensions often formed along the edge of the cell (for review, see Hall, 1998, Science 279, 509-514). In brain, small G proteins participate in the morphological changes of neurons, localized in growth cones, axons, dendritic trunks, and spines (van Leeuwen, F. N., van Delft, S., Kain, H. E., van der Kammen, R. A., and Collard, J. G. (1999) Nat. Cell Biol. 1, 242-248). In the AD brain, neuronal Cdc42/Rac are upregulated in select neuronal populations in comparison to age-matched controls, in relation to the pathogenic process and neuronal degeneration (Zhu, X., Raina, A. K., Boux, H., Simmons, Z. L., Takeda, A., and Smith, M. A. (2000) Int. J. Dev. Neurosci. 18, 433-437). In the mature brain, Rac1, but not Rho nor Cdc42, is present in the raft domain of neuronal membranes (Kumanogoh, H., Miyata, S., Sokawa, Y., and Maekawa, S. (2001) Neurosci. Res. 39, 189-196). In addition, a recent unbiased quantitative proteomics study revealed Rac1 as a raft-associated protein (Foster, L. J., De Hoog, C. L., and Mann, M. (2003) Proc. Natl. Acad. Sci USA 100, 5813). Other studies showed that activation of Rac1 is associated with its rapid recruitment into the lipid rafts while Cdc42 is not recruited into rafts, but activated by raft-associated moieties and, more important, that Rac1, but not Rho nor Cdc42, regulates the assembly and export to the cell membrane of Golgi-derived lipid rafts (Field, K. A., J. R. Apgar, E. Hong-Geller, R. P. Siraganian, B. Baird, and D. Holowka. (2000) Mol. Biol. Cell 11, 3661; Rozelle, A. L., L. M. Machesky, M. Yamamoto, M. H. Driessens, R. H. Insall, M. G. Roth, K. Luby-Phelps, G. Marriott, A. Hall, and H. L. Yin. 2000 Curr. Biol. 10:311).

Rac1b is a particular splicing form of Rac1, wherein exon3b is retained. The sequence of Rac1b is provided in the present application, as well as the sequence of exon3b (see nucleotides 51-170 of SEQ ID NO: 12). These sequences are also available in public gene libraries.

Within the context of the invention, the term Rac1b RNA denotes any RNA sequence, either coding or non-coding, whether mature or not, that eventually results in the expression of a Rac1b polypeptide.

The term “Rac1 gene” denotes any nucleic acid encoding a Rac1 polypeptide. It can be genomic (gDNA), complementary (cDNA), synthetic or semi-synthetic DNA, mRNA, synthetic RNA, etc. It can be a recombinant or synthetic nucleic acid, produced by techniques known to those skilled in the art, such as artificial synthesis, amplification, enzymatic cleavage, ligation, recombination, etc., using biological sources, available sequences or commercial material. A Rac1 gene exists typically in a two-stranded form, even though different forms can exist according to the invention. The sequence of the Rac1 gene is available in certain data banks, such as, notably, RefSeq, No. NM_{—}009007. Other Rac1 gene sequences, according to the invention, can be isolated from samples, or collections, or may be synthesized.

The term Rac1b polypeptide particularly denotes any Rac1 polypeptide comprising amino acids encoded by exon3b. The term Rac1b polypeptide also includes, in the broad sense, any biologically active natural variant of the sequence identified above, resulting from e.g., polymorphisms, mutations, insertions, etc.

### Rac1b Inhibitors

Within the context of this invention, the term “Rac1b inhibitor” designates any compound or treatment that reduces or blocks the activity or expression of Rac1b. More preferred Rac1b inhibitors are compounds that inhibit Rac1b-dependent cytoskeleton rearrangements. Most preferred Rac1b inhibitors are selective inhibitors, e.g., they are able to bind to or react with Rac1b-specific domains, particularly all or part of exon3b. Preferred Rac1b inhibitors essentially do not directly affect cdc42 and/or RhoA, i.e., do not substantially interact with cdc42 and/or RhoA, respectively.

Specific examples of such Rac1b inhibitors include any compound that specifically binds Rac1b or a domain thereof, particularly exon3b or a domain thereof. Such binding compounds include, for instance, any inhibitory nucleic acid, such as antisense RNA, ribozyme, iRNA, siRNA, ssRNA, microRNAs, etc., or any corresponding DNA, PNA, etc. Such inhibitory nucleic acids preferably comprise a sequence that is complementary to all or a portion of exon3b of Rac1 gene, thereby preventing, blocking or reducing the transcription or translation thereof in a cell. Specific examples of such inhibitory oligonucleotides are disclosed below in this application.

Another example of such Rac1b inhibitors is an antibody (or a fragment or derivative thereof), that binds Rac1b. Such an antibody typically binds an epitope comprised within amino acids encoded by exon3b of Rac1. The antibody may be polyclonal or, preferably, monoclonal. Antibody fragments include, e.g., Fab fragments, Fab′2 fragments, CDR regions, etc. Antibody derivatives include single chain antibodies, humanized antibodies, human antibodies, bifunctional antibodies, etc.

A particular Rac1b inhibitor is an antibody, preferably a monoclonal antibody (or a derivative or fragment thereof), that specifically binds an epitope comprised within (i.e., fully or partially included within) exon3b of Rac1b. Even more preferably, the invention relates to an antibody, preferably a monoclonal antibody (or a derivative or fragment thereof), that specifically binds an epitope comprised within SEQ ID NO: 13.

A further aspect of this invention is an antibody, preferably a monoclonal antibody (or a derivative or fragment thereof, such as a CDR sequence), that specifically binds Rac1b, produced by immunization of a non human mammal with a polypeptide comprising SEQ ID NO: 13. A further object of this invention is a polypeptide comprising SEQ ID NO: 13 or an epitope-containing fragment thereof of at least 5 contiguous amino acids. More specifically, an object of this invention is a polypeptide of less than 50 amino acids in length, comprising SEQ ID NO: 13 or an epitope-containing fragment thereof of at least 5 contiguous amino acids.

Antibodies against human Rac1b protein may be produced by procedures generally known in the art. For example, polyclonal antibodies may be produced by injecting the protein alone or coupled to a suitable protein into a non-human animal. After an appropriate period, the animal is bled, sera recovered and purified by techniques known in the art (see Paul, W. E. “Fundamental Immunology” Second Ed. Raven Press, NY, p. 176, 1989; Harlow et al “Antibodies: A laboratory Manual”, CSH Press, 1988; Ward et al (Nature 341 (1989) 544). Monoclonal antibodies may be prepared, for example, by the Kohler-Millstein technique (Kohler-Millstein, Galfre, G., and Milstein, C, Methods Enz. 73 p. 1 (1981)) involving fusion of an immune B-lymphocyte to myeloma cells. For example, an antigen as described above can be injected into a suitable non-human mammal (e.g., a mice) until a polyclonal antibody response is detected in the serum. The mammal can be boosted again, its spleen removed and fusion with myeloma conducted according to a variety of methods. The individual surviving hybridoma cells can be tested for the secretion of anti-rac1b antibodies first by their ability to bind the immunizing antigen and then by their ability to immunoprecipitate rac1b from cells.

### Therapeutic Uses

A particular object of this invention relates to a method of treating a degenerative disorder in a mammalian subject, comprising administering to a subject in need thereof an effective amount of a Rac1b inhibitor. Preferably, the compound is administered in an amount effective at reducing APP processing in said subject.

A further aspect of this invention is a method of inhibiting the generation of an amyloïd beta peptide in a mammalian subject, comprising administering to a subject in need thereof an amount of a Rac1b inhibitor effective at reducing APP processing in said subject.

A further aspect of this invention is a method of inhibiting the generation of an amyloïd beta peptide in a mammalian subject without substantially altering the Notch cleavage or BACE activity, comprising administering to a subject in need thereof an effective amount of a Rac1b inhibitor.

A particular object of this invention relates to a method of treating Alzheimer's diseased disorder in a mammalian subject, comprising administering to a subject in need thereof an amount of a Rac1b inhibitor effective at reducing APP processing in said subject.

A further aspect of this invention is a method of inhibiting the generation of an amyloïd beta peptide in a mammalian subject without substantially altering the Notch cleavage or BACE activity, comprising administering to a subject in need thereof an effective amount of a Rac1b inhibitor.

For use in the present invention, the compounds may be in the form of a pharmaceutical composition comprising at least one of said compounds and a pharmaceutically acceptable vehicle or support. The compounds may be formulated in various forms, including solid and liquid forms, such as capsules, tablets, gel, solution, syrup, suspension, powder, aerosol, ointment, etc.

Such pharmaceutical compositions of this invention may contain physiologically acceptable diluents, fillers, lubricants, excipients, solvents, binders, stabilizers, and the like. Diluents that may be used in the compositions include but are not limited to dicalcium phosphate, calcium sulphate, lactose, cellulose, kaolin, mannitol, sodium chloride, dry starch, powdered sugar and for prolonged release tablet-hydroxy propyl methyl cellulose (HPMC). The binders that may be used in the compositions include but are not limited to starch, gelatin and fillers such as sucrose, glucose, dextrose and lactose.

Natural and synthetic gums that may be used in the compositions include but are not limited to sodium alginate, ghatti gum, carboxymethyl cellulose, methyl cellulose, polyvinyl pyrrolidone and veegum. Excipients that may be used in the compositions include but are not limited to microcrystalline cellulose, calcium sulfate, dicalcium phosphate, starch, magnesium stearate, lactose, and sucrose. Stabilizers that may be used include but are not limited to polysaccharides such as acacia, agar, alginic acid, guar gum and tragacanth, amphotsics such as gelatin and synthetic and semi-synthetic polymers such as carbomer resins, cellulose ethers and carboxymethyl chitin.

Solvents that may be used include but are not limited to Ringers solution, water, distilled water, dimethyl sulfoxide to 50% in water, propylene glycol (neat or in water), phosphate buffered saline, balanced salt solution, glycol and other conventional fluids.

The dosages and dosage regimen in which the compounds are administered will vary according to the dosage form, mode of administration, the condition being treated and particulars of the patient being treated. Accordingly, optimal therapeutic concentrations will be best determined at the time and place through routine experimentation.

The compounds according to the invention can also be used enterally. Orally, the compounds according to the invention are suitable administered at the rate of 10 μg to 300 mg per day per kg of body weight. The required dose can be administered in one or more portions. For oral administration, suitable forms are, for example, capsules, tablets, gel, aerosols, pills, dragees, syrups, suspensions, emulsions, solutions, powders and granules; a preferred method of administration consists in using a suitable form containing from 1 mg to about 500 mg of active substance.

The compounds according to the invention can also be administered parenterally in the form of solutions or suspensions for intravenous, subcutaneous or intramuscular perfusions or injections. In that case, the compounds according to the invention are generally administered at the rate of about 10 μg to 10 mg per day per kg of body weight; a preferred method of administration consists of using solutions or suspensions containing approximately from 0.01 mg to 1 mg of active substance per ml.

The compounds according to the invention can also be administered in the eye in the form of solutions or suspensions for intravitreous or retro-orbitary injections. In that case, the compounds according to the invention are generally administered at the rate of about 10 μg to 10 mg per day per kg of body weight; a preferred method of administration consists of using solutions, suspensions or gel containing approximately from 0.01 mg to 1 mg of active substance per ml.

The compounds can be used in a substantially similar manner to other known agents for treating CNS disorders. The dose to be administered, whether a single dose, multiple dose, or a daily dose, will vary with the particular compound employed because of the varying potency of the compound, the chosen route of administration, the size of the recipient, the type of disease and the nature of the patient's condition. The dosage to be administered is not subject to definite bounds, but it will usually be an effective amount, or the equivalent on a molar basis of the pharmacologically active free form produced from a dosage formulation upon the metabolic release of the active drug to achieve its desired pharmacological and physiological effects. A physician or a doctor skilled in the art of CNS disorder treatment will be able to ascertain, without undue experimentation, appropriate protocols for the effective administration of the compounds of this invention.

The compounds may be administered according to various routes, typically by oral route or by injection, such as local or systemic injection(s). Oral, intravenous, intraperitoneal or sub-cutaneous administration are preferred, although other administration routes may be used as well, such as intramuscular, intradermic, etc. Furthermore, repeated injections may be performed, if appropriate.

A further aspect of this invention relates to the use of a Rac1b inhibitor for the manufacture of a pharmaceutical composition for inhibiting the generation of an amyloïd beta peptide in a mammalian subject having a degenerative disorder, particularly Alzheimer's disease.

A further object of this invention is the use of a Rac1b inhibitor for the preparation of a pharmaceutical composition for treating Alzheimer's disease.

### Drug Screening

The invention implicates, for the first time, Rac1b in the modulation of APP processing and Aβ generation. Accordingly, the invention shows that Rac1b represents a valuable target for the screening of drugs to be used in the treatment of degenerative diseases.

In this respect, a particular object of this invention relates to methods of producing, identifying, selecting or optimising candidate compounds for use in the treatment of amyloïd beta peptide-related disorders, the method comprising determining whether a test compound inhibits Rac1b, Rac1 inhibition being an indication that the test compound is a candidate compound for use in the treatment of amyloïd beta peptide-related disorders. Rac1b inhibition may be assessed in vitro, ex vivo or in vivo, according to various biological assays which are known per se in the art.

In a particular embodiment, the method comprises contacting the test compound and Rac1b (or a fragment thereof, typically comprising all or part of exon3b) and determining whether the compound binds Rac1b or the fragment thereof.

In another particular embodiment, the method comprises contacting the test compound and Rac1b and determining whether the compound inhibits Rac1b-dependent cytoskeleton rearrangements.

In a particular embodiment, Rac1b inhibition is assessed using the effector PAK1 pull-down assay.

More preferably, the compounds are further assessed for their activity towards other targets, particularly the Notch processing pathway (e.g., Notch cleavage), BACE, or other small GTP-binding proteins (e.g., Cdc42 and/or RhoA). Most preferred compounds are those which substantially do not alter Notch cleavage and/or do not substantially directly inhibit BACE, and/or do not substantially directly inhibit Cdc42 and/or RhoA.

The assays may be conducted in vitro, in any suitable device, and various test compounds may be assayed in parallel, or in mixtures.

### Diagnostic

The present invention further relates to methods of detecting the presence or predisposition to oxidative stress comprising detecting, in a sample from a subject, the presence or (relative) amount of Rac1b, the presence of Rac1b being indicative of the presence or predisposition to oxidative stress.

The present invention further relates to methods of detecting the presence, stage or predisposition to a degenerative disorder in a subject, comprising detecting, in a sample from the subject, the presence or (relative) amount of Rac1b, the presence of Rac1b being indicative of the presence, stage or predisposition to said disorder.

In more preferred embodiments, the above methods comprise measuring the ratio of Rac1a and Rac1b isoforms, a variation within said ratio being an indication as to the predisposition, presence or stage of the disease. More specifically, the method comprises detecting the presence of a nucleic acid molecule comprising any one of SEQ IS NO: 1-8 or 12, or a complementary strand thereof, or a corresponding polypeptide. Such nucleic acid molecules and polypeptides also represent particular object of the present invention, as well as any distinctive fragment or analogs thereof; antibodies specifically binding to such polypeptides and specific nucleic acid probes or primers.

Detection can be performed according to techniques known per se in the art, such as amplification, hybridization, sequencing, immunological techniques, etc. In this respect, the invention discloses particular oligonucleotides, which specifically distinguish between Rac1a and Rac1b, and represent a particular object of this invention. Such oligonucleotides preferably bind a junction region created by splicing of exon3b or by retention of exon3b, and/or bind to exon 3b itself. Examples of such oligonucleotides are provided in SEQ ID NO: 9-11. This invention encompasses any oligonucleotide (preferably single stranded, comprising between 5 and 60 bases, more preferably 5-50, 5-40, 10-30, 10-25), that specifically binds Rac1 exon3b or a junction region created by retention or splicing or said exon3b.

The complete human genomic sequence of Rac1 is available at NCBI under accession number AJ132695. Exon 3b corresponds to nucleotide positions 18413-18530 of said sequence, which is reproduced below (SEQ ID NO: 12), from positions 18361 to 18600 (exon 3b is in bold):

18361 cagtgactta gcttctacac ctgtgactaa ccattttcat tccattctac agctttgaca
18421 attattctgc caatgttatg gtagatggaa aaccggtgaa tctgggctta tgggatacag
18481 ctggacaaga agattatgac agattacgcc ccctatccta tccgcaaaca gtaaggattg
18541 cagctgactt ttaatgtgtc ttttagagta tataattctc gagcgcttaa ttagtgcatg

Specific oligonucleotides of this invention comprise a sequence that specifically hybridises to a junction region between Rac1b exon 3b and flanking sequences, within RNA molecules. Such oligonucleotides typically comprise a domain of at least 5 nucleotides which is specific for a 5′ or 3′ end of exon 3b, directly fused to a domain of at least 5 nucleotides which is specific for a sequence flanking said 5′ or 3′ end of exon 3b, respectively. Alternative oligonucleotides of this invention specifically hybridise to the junction regions created by splicing of exon3b, i.e., to the following target junction: tctacgtaag. Such oligonucleotides may be used as primers or as probes, to amplify or detect the presence and/or (relative) amount of Rac1b in a sample, and represent particular object of this application. Other specific oligonucleotides of this invention specifically hybridise to a sequence contained within exon3b (i.e., within nucleotide residues 51-170 of SEQ ID NO: 12).

Further aspects and advantages of this invention will be disclosed in the following examples, which should be regarded as illustrative and not limiting the scope of this application. All cited publications or applications are incorporated therein by reference in their entirety.

### EXAMPLES

### Example 1

### Identification of rac1b DATAS Signatures from AD Brain

The DATAS technology for expression profiling studies (DATAS U.S. Pat. No. 6,251,590) was used to compare alternative RNA splicing events present in the prefrontal cortex of well characterized AD patients and healthy controls. Control subjects were non demented, were age-matched and had similar post-mortem delay to filter out age-related and mRNA stability-related changes in profiling studies.

### Identification of an Alteration of Rac1

Among the clones identified were 5 fragments of mRNA corresponding to a RAS-related C3 botulinum substrate 1 (Rac1), indicating a dysregulation of the splicing events of rac1 in the brain of patients suffering from AD.

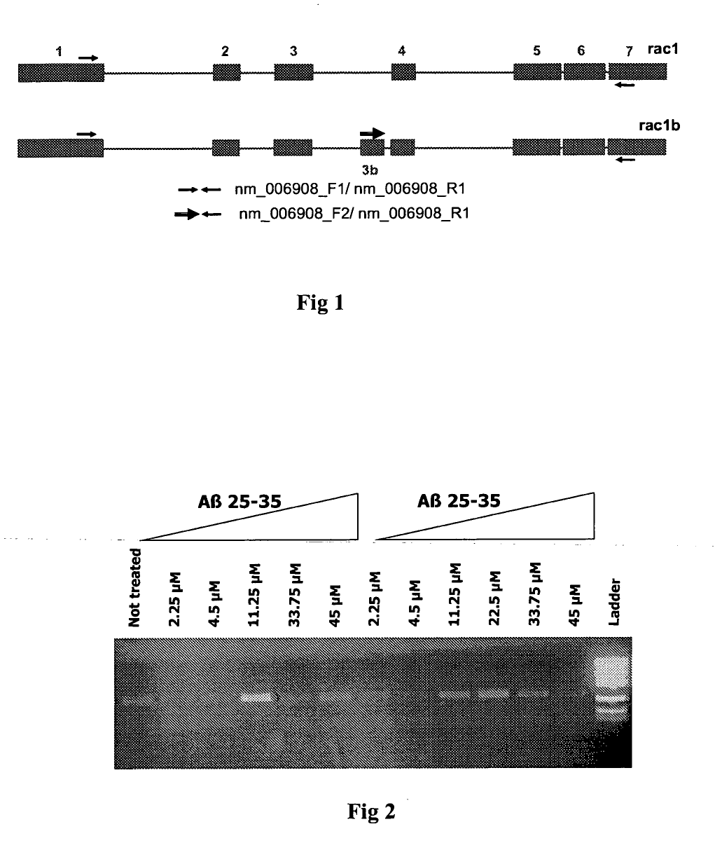

The DATAS fragments are: EXH-NADC4422-01, length: 354 (SEQ ID NO1), EXH-NADC4506-01 (SEQ ID NO 2), length 344, EXH-NADC4513-01, length 354 (SEQ ID NO 3), EXH-NADC4524-01, length 354 (SEQ ID NO4), EXH-NADC4531-01, length 348 (SEQ ID NO 5), EXH-NADC4534-01, length 356 (SEQ ID NO 6), and EXH-NADC4550-01, length 353 (SEQ ID NO 7). These DATAS fragments organize in a cluster, cluster13983_{—}2 (SEQ ID NO 8) corresponding to nucleotides 246 to 782 of the RefSeq bank sequence, referenced under the number NM_{—}006908. This region includes the alternatively spliced 57 bp region (exon 3b) that is present in transcript variant Rac1b referenced in RefSeq bank sequence as NM_{—}018890 (FIG. 1).

### Example 2

### Induction of Ra1b Splicing by Abeta

Rat primary cortical cell cultures are sensitive to Abeta toxicity, the neurotoxicity of which involves different mechanisms such as calcium homeostasis dysregulation, accumulation of ROS, secondary excitotoxicity and caspase activation (Zhang et al, J Neurochem 1996, 67 (4) 1595-1606). Abeta peptide is dissolved in 100% HFIP (1,1,1,3,3,3-hexafluoro-2-propanol), then dryed out and resuspended in 100% DMSO and rediluted in DMEM/F12 without phenol red. The solution maturates by incubation at 37° C. during 72 h and Aβ oligomers are then added on 7 days cultures for 24 h. After 24 h incubation, cells were processed for RNA extraction using Trizol. A nested PCR approach was used for the amplification rac1b using the following primers: nm_{—}006908_F1: ATGCAGGCCATCAAGTGTGTGG (SEQ ID NO 9), nm_{—}006908_R1: TGGCATTGAGTGCGAAGGC (SEQ ID NO 10), nm_{—}006908_F2: AAAGACAAGCCGATTGCCG (SEQ ID NO 11). Rac1 was amplified following the first PCR of the protocol described below using primers nm_{—}006908_F1 and nm_{—}006908_R1. In these conditions, rac1b was not detected on gel due to its low quantity after single PCR amplification and was easily detected using primers nm_{—}006908_F2 and nm_{—}006908_R1 in the nested PCR.

Reverse transcription (Transcriptor Reverse Transcriptase, Roche) was performed using Random primers p(dN)₆ (Invitrogen), 1 mM dNTP mixture, 1 unit/μl RNAse OUT ribonuclease Inhibitor (Invitrogen) and 0.5 units/μl Transcriptor reverse Transcriptase. First PCR was conducted using Platinum® Taq DNA Polymerase High Fidelity (Invitrogen) with 0.2 mM dNTP mixture, 1.5 mM MgSO4, 0.25 μM of each primer (nm_{—}006908_F1 and nm_{—}006908_R1) and 0.1 unit/μl Platinum® Taq High Fidelity for 30 cycles of amplification as follows: denaturation: 94° C. for 30 s, annealing: 60° C. for 1 min, extension 68° C. for 1 min. PCR amplicon was purified using MicroSpin™ S-300 HR Columns (Amersham Biosciences) and a second PCR was conducted using PCR Master Mix (Promega) and 0.2 μM of each primers (nm_{—}006908_F2 and nm_{—}006908_R1) for 30 cycles of PCR amplification as follows: denaturation 94° C. for 30 s, annealing 60° C. for 1 min, extension 72° C. for 1 min. PCR products were resolved on 1.5% agarose gel.

### Results

To test the effect of Abeta treatment on mRNA levels of rac1b, cortical cells were exposed to various Abeta concentrations for 24 hours and rac1b was specifically amplified in a nested PCR approach that sequentially used primers nm_{—}006908_F1 and nm_{—}006908_R1, then primers nm_{—}006908_F2 and nm_{—}006908_R1. GAPDH was amplified as control. Results obtained in two independent cultures indicate a dose dependent accumulation of rac1b mRNA observed at the concentration of 11.25 and 22.5 μM Abeta (FIG. 2). This indicates that Abeta alters the rac1/rac1b ratio so that rac1 splicing is biased towards the incorporation of an alternative exon 3b. Thus, low level of Abeta stimulates the appearance of a highly activated variant of Rac1, predominantly GTP-bound, with a selective downstream signalling.

### Example 3

### Induction of Rac1b Splicing by Oxidative Stress

SKNSH sub-clone SH-SY5Y (ATCC, CRL-2266) is a widely accepted model to study oxidative stress mediated neurotoxicity, or neuroprotection (Zuo et al. 1995).

The organic hydroperoxide t-butyl hydroperoxide (TBH) is a stable analogue of H₂O₂. TBH induces apoptosis following a sequence of events that are initiated by ROS generation, loss of redox imbalance, mitochondrial cytochrome c release, and activation of caspase-3.

The catecholamine-specific neurotoxin 6-hydroxydopamine (6-OHDA) classically used to create animal models of Parkinson's disease is a hydroxylated analogue of dopamine that leads to apoptosis of catecholaminergic cells. This neurotoxin induces apoptosis and its toxicity is associated with ROS production. Several mechanisms are probably involved: (1) reactive-oxygen ROS generation by auto-oxidation, (2) hydrogen peroxide generation after deamination by monoamine oxidase, (3) direct inhibition of mitochondrial complexes I and IV and protein degradation and ubiquitin-proteasome system activation (Youdim et al. 2001).

To test whether ROS production may affect the rac1/rac1b ratio SH-SY5Y cells were exposed to various ROS-inducing agents at the indicated concentrations for 24 hours and cell viability was determined using a LDH assay (Cytotox96, Promega).

SH-SY5Y cells were plated in 24 well plates (ATGC, France) at the initial density of 3*10⁵ cells/well. After 24 hours, cells were treated with various ROS-inducing agents at the indicated concentrations. After 24 h incubation, a LDH assay was conducted to reveal cell viability and cells were processed for RNA extraction using Trizol.

### Results

To test the effect of ROS producing agents TBH and 6-OHDA on mRNA levels of rac1 and rac1b, SH-SY5Y cells were exposed to various ROS-inducing agents at the indicated concentrations for 24 hours and rac1 and rac1b were either coamplified using primers nm_{—}006908_F1 and nm_{—}006908_R1, or rac1b was specifically amplified in a nested PCR approach that sequentially used primers nm_{—}006908_F1 and nm_{—}006908_R1, then primers nm_{—}006908_F2 and nm_{—}006908_R1. GAPDH was amplified as control.

In parallel, a LDH assay to monitor cell viability on the same cells.

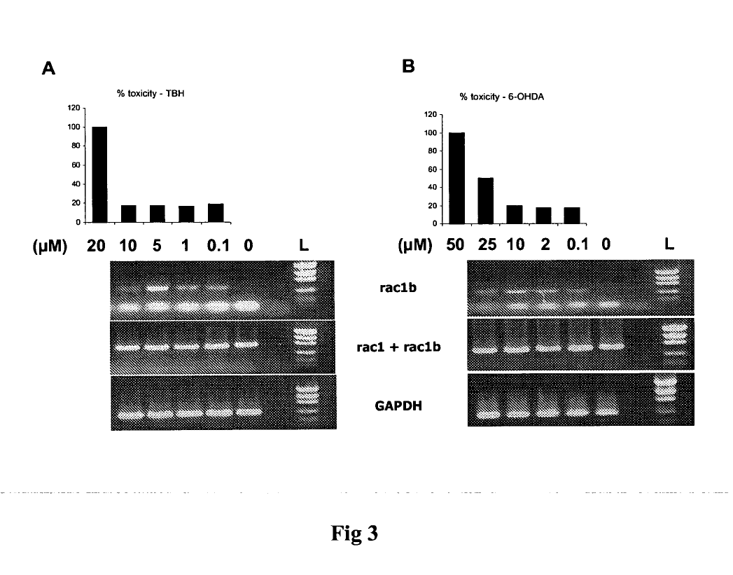

Results indicate that for both toxics, rac1 levels are not altered upon oxidative stress (FIG. 3). In contrast, a dose dependent accumulation of rac1b is observed when subtoxic concentrations of both TBH and 6-OHDA are used. This indicates that low levels of oxidative stress also alter the rac1/rac1b ratio so that rac1 splicing is biased towards the incorporation of alternative exon 3b, thus stimulating the appearance of the highly activated variant of Rac1, Rac1b.

1. A method of treating a degenerative disorder in a mammalian subject, comprising administering to a subject in need thereof an effective amount of a Rac1b inhibitor.

2. A method of inhibiting the generation of an amyloïd beta peptide in a mammalian subject, comprising administering to a subject in need thereof an effective amount of a Rac1b inhibitor.

3. A method of claim 1, wherein the compound does not substantially alter Notch cleavage or BACE activity.

4. The method of claim 1, for treating Alzheimer's disease.

5. A method of producing, identifying, selecting or optimising candidate compounds for use in the treatment of degenerative disorders, the method comprising determining whether a test compound inhibits Rac1b, Rac1b inhibition being an indication that the test compound is a candidate compound for use in the treatment of degenerative disorders.

6. The method of claim 5, comprising contacting the test compound and Rac1b and determining whether the compound binds Rac1b or inhibits Rac1b-dependent cytoskeleton rearrangements.

7. A method of detecting the presence or predisposition to oxidative stress comprising detecting, in a sample from a subject, the presence or (relative) amount of Rac1b, the presence of Rac1b being indicative of the presence or predisposition to Aβ-related oxidative stress.

8. A method of detecting the presence, stage or predisposition to a degenerative disorder in a subject, comprising detecting, in a sample from the subject, the presence or (relative) amount of Rac1b, the presence of Rac1b being indicative of the presence, stage or predisposition to said disorder.

9. The method of claim 8, comprising measuring the ratio of Rac1a and Rac1b isoforms, a variation within said ratio being an indication as to the predisposition, presence or stage of the disease.

10. An oligonucleotide or a set of oligonucleotides, which specifically distinguishes between Rac1a and Rac1b.

11. A single stranded oligonucleotide, comprising between 5 and 60 bases, that specifically binds Rac1 exon3b or a junction region created by retention or splicing or said exon3b.

12. An antibody that binds an epitope comprised within
VGETYGKDITSRGKDKPIA. (SEQ ID NO: 13)

13. A polypeptide of less than 50 amino acids in length, comprising SEQ ID NO: 13 or an epitope-containing fragment thereof of at least 5 contiguous amino acids.
